(19) United States
(12) Patent Application Publication
(10) Pub. No.: US 2025/0366801 A1
(43) Pub. Date: Dec. 4, 2025

(54) ESTIMATION OF CEREBROVASCULAR RESERVE

(71) Applicant: MedTrace Pharma A/S, Hørsholm (DK)

(72) Inventors: Hans Harms, Hørsholm (DK); Mark Lubberink, Hørsholm (DK)

(73) Assignee: MedTrace Pharma A/S, Hørsholm (DK)

(21) Appl. No.: 19/216,124

(22) Filed: May 22, 2025

(30) Foreign Application Priority Data
May 29, 2024 (EP) 24178667.2
Mar. 21, 2025 (EP) 25165224.4

(51) Int. Cl. A61B 6/03; A61B 6/00; A61B 6/50; A61B 6/58

(52) U.S. Cl. CPC A61B 6/037; A61B 6/486; A61B 6/501; A61B 6/58; A61B 2576/026

(57) ABSTRACT

A method for non-invasive quantitative measurement of cerebrovascular reserve (CVR) and cerebral blood flow (CBF) with ¹⁵O-water positron emission tomography (PET) in a human.

### CROSS REFERENCE TO RELATED APPLICATIONS

The present application is based on and claims priority to European Patent Application Serial Number 24178667.2, filed May 29, 2024, and European Patent Application Serial Number 25165224.4, filed Mar. 21, 2025, the disclosures of which are incorporated herein by reference.

### FIELD OF THE INVENTION

The present invention relates to a method for non-invasive and non-therapeutic quantitative measurement of cerebrovascular reserve (CVR) and cerebral blood flow (CBF) with 15O-water positron emission tomography (PET).

### BACKGROUND OF THE INVENTION

Evaluation of cerebral perfusion is important for patients with cerebrovascular disease to diagnose the hemodynamic significance of vascular lesions and determine the optimal treatment tactics for the patient. Cerebrovascular reserve capacity (CVR) describes how far cerebral perfusion can increase from a baseline value after stimulation.

The reserve of cerebral blood flow (CBF) can be estimated by measuring cerebrovascular reactivity to external stimuli (Gupta 2012). There have been two main approaches to measuring CVR. One approach attempts direct cerebral blood flow measurements of the brain tissue with flow-sensitive imaging techniques such as positron emission tomography, nuclear medicine techniques, CT perfusion, or MR perfusion before and after a vasodilatory stimulus. The second approach involves transcranial Doppler measurement of flow velocities (typically in the middle cerebral artery) distal to a lesion both before and after a vasodilatory stimulus with the increase flow velocity considered a surrogate for CVR.

Vasodilatory stimuli include increasing levels of CO2 (such as with breath-holding or inhalation of CO2 gas mixtures) and pharmacological challenge in the form of various pharmacological agents possessing vasoactive properties. These drugs include acetazolamide (an inhibitor of carbonic anhydrase), which causes an acidosis and a significant increase in brain perfusion due to dilatation of intracranial arteries. Acetazolamide causes a decrease in the resistance of cerebral vessels, accompanied by an increase in CO2 level and corresponding decrease in pH in the blood.

Acetazolamide increases cerebral blood flow markedly in unaffected vessels, whereas in areas where blood is supplied by stenotic or malformed vessels, the flow either increases slightly or remains unchanged (Mamontov 2020). The so-called “acetazolamide challenge test” is used routinely in patients with atherosclerotic cerebrovascular disease using a single, standard dose of 1000 mg (1 g) administered intravenously. This dosage serves a purely diagnostic purpose in the acetazolamide challenge test to assess cerebrovascular reactivity and reserve. The peak cerebral blood flow augmentation occurs approximately 10-15 minutes after the intravenous bolus injection (Vagal 2009).

Apart from the diagnostic use in connection with testing for atherosclerotic cerebrovascular disease, acetazolamide has several therapeutic uses, including:
Glaucoma treatment
Epilepsy management
Congestive heart failure and edema treatment
Altitude sickness prevention and treatment
Idiopathic intracranial hypertension

The dosage and dosing schedule vary depending on the condition being treated. For example, the dose range recommended for the treatment of glaucoma is 250 to 1000 mg per day. For treating altitude sickness, the range is 250 to 500 mg daily in 2 oral doses (Farzam 2023). By far the most common therapeutic use of acetazolamide involves administering repeated, often daily doses of acetazolamide over a period of time, regardless of the treated disease, except for a few rare cases where an acute medical condition may be alleviated with a single dose of acetazolamide. For therapeutic purposes, a single dose of acetazolamide is thus considered relevant only for acute medical conditions.

In a systematic review and meta-analysis (Gupta 2012) of 1061 independent CVR tests in 991 unique patients with carotid stenosis or occlusion with a mean follow-up of 32.7months, baseline CVR impairment was associated with increased risk of stroke or transient ischemic attack (TIA). The findings suggest a positive relationship between baseline CVR impairment and future ischemic events with a pooled odds ratio suggesting that patients with impaired CVR are approximately four times more likely to develop stroke or TIA.

The gold standard for in-vivo CBF measurements is positron emission tomography (PET) with 15O-water, since 15O-water is freely diffusible, metabolically inert and has an uptake rate that is linear with blood flow up to high flow values. Measurement of CBF with 15O-water requires the acquisition of an arterial input function, which can only be obtained accurately using radioactivity measurements of continuously sampled blood from an indwelling arterial catheter, usually placed in the radial artery. This is an invasive, not completely risk-free and often painful procedure, which has hampered the clinical application of this method (Chim 2015, Mandel 1977, Wallach 2004, Everett 2009). As a result, measurement of CBF with 15O-water is not used often, and potentially critical diagnostic information is therefore not routinely obtained.

Hence, the availability of a safe, non-invasive, but still fully quantitative method to determine CBF and CVR using 15O-water and PET would enable widespread clinical use of quantitatively accurate CBF and CVR measurements.

### BRIEF DESCRIPTION OF DRAWINGS

FIG. 1—Correlation (top) and agreement (bottom) between true CVR and measured CVR using noise-free simulated data, using the 3-parameter implementation of equation 9 (left) and the corresponding 2-parameter implementation (right).

FIG. 2—Correlation (top) and agreement (bottom) between true CVR and measured CVR using noisy simulated data, using the 3-parameter implementation of equation 9 (left) and the corresponding 2-parameter implementation (right).

FIG. 3—Correlation (top) and agreement (bottom) between true CVR and measured CVR using noise-free simulated data including a 5% partial blood volume, using the 3-parameter implementation of equation 9 (left) and the corresponding 2-parameter implementation (right).

FIG. 4—Correlation (top) and agreement (bottom) between true CVR and measured CVR using noisy simulated data including a 5% partial blood volume, using the 3-parameter implementation of equation 9 (left) and the corresponding 2-parameter implementation (right).

FIG. 5—Correlation (top) and agreement (bottom) between true CVR and measured CVR using noisy simulated data including a 5% partial blood volume, using the 3-parameter implementation of equation 9 (left) and the corresponding 2-parameter implementation (right). Blood volume was now accounted for in the models by subtracting a noisy version of the original blood curve.

FIG. 6—Correlation (top) and agreement (bottom) between true CVR and measured CVR using noisy simulated data including a 5% partial blood volume, using the 3-parameter implementation of equation 9 (left) and the corresponding 2-parameter implementation (right).

FIG. 7—Typical fit of the proposed model to a total brain grey matter baseline time-activity curve. Three-(3p) and two-parameter (2p) fits overlap.

FIGS. 8-8A: CVR measured using the 2-parameter non-invasive model versus CVR measured using the invasive method. FIG. 8A is equal to “FIG. 8—Top” in the priority document EP24178667.2. B: As 8A, but after scaling the post-acetazolamide time-activity curves by the ratio of the areas under the curve of the first-pass phases of the baseline. C: As 8B, but after scaling the post-acetazolamide time-activity curves by the ratio of the areas under the curve of the first-pass phases of the baseline and post-acetazolamide arterial input functions, to correct for differences in injected activity and post-acetazolamide arterial input functions, to correct for differences in injected activity. FIG. 8C thus indicates what could be achieved with a point-of-care production system (e.g. medtracepharma.com) with a highly reproducible bolus administration in terms of amount of activity, volume, and injection speed. The solid lines are orthogonal regression fits, the dashed lines are lines of identity (x=y). Closed circles represent original data points; open circles represent healthy subjects included after filing EP24178667.2.

### SUMMARY OF THE INVENTION

It has now surprisingly been found that the need for arterial cannulation and sampling to obtain quantitative CBF and CVR values with 15O-water can be obviated by a method which comprises two 15O-water PET scans based on a fixed amount of radioactivity being administered at a fixed injection rate.

It is known how to describe the tracer kinetic analysis of 15O-water using differential equations. Rearranging the differential equations describing the tracer kinetic analysis of 15O-water at baseline and during vasodilation, yields, when injection of the radioactivity is done in a controlled and reproducible manner, a solution, where the arterial input function is no longer required. Instead, the vasodilation data is described as a function of the baseline data and CVR, or vice-versa. Together with a controlled automated power injector, injecting a fixed amount of radioactivity at a fixed injection rate, the method allows for non-invasive measurement of CVR and CBF using 15O-water and PET. The method is valid using any intervention that changes blood flow, but only blood flow, between the two PET scans.

According to the method of the present invention, a 1st known amount of 15O -water is administered to the patient using a controlled bolus injection. A first PET scan of the brain is simultaneously initiated. After a period of about 15 minutes, a 2nd controlled bolus injection of a known amount of 15O-water is administered, and a second PET scan of the brain is simultaneously initiated. Azetazolamide (10 mg/kg) or another vasodilatant is administered according to the acetazolamide challenge test conditions approximately 15 minutes before the start of the second PET scan (but after the end of the first PET scan), which administration serves a purely diagnostic purpose as dicussed hereinabove.

After the second PET scan has been acquired, both PET acquisitions are reconstructed into a dynamic series using all appropriate corrections for quantitative PET. The activity concentrations in identical regions of interest in both scans are then measured and time-activity curves (TACs) are constructed showing the activity concentration over time. Finally, the combinations of TACs are fitted to a tracer kinetic model that describes one of the TACs as a function of the other.

Accordingly, in a first aspect of the invention there is provided a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1st PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/V_{T} (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),
   wherein the start of the two PET acquisitions of steps a) and c) are interspaced by at least 6 half-lives of 15O (about 12 min).

In a second aspect of the invention acetazolamide is provided for use in a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/V_{T} (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),
   wherein the start of the two PET acquisitions of steps a) and c) are interspaced by at least 6 half-lives of 15O (about 12 min).

In a third aspect of the invention there is provided a bolus composition comprising a first IV bolus 15O-water and a second IV bolus 15O-water, for use in a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/VT (where VT is the water partition coefficient) and CVR,
g) returning estimated (maximum-likelihood) parameter values of the pharmacokinetic model for the brain including cerebral blood flow (CBF) and an estimate of the cerebrovascular reserve (CVR),

wherein the start of the two PET acquisitions of steps a) and c) are interspaced by at least 6 half-lives of 15O (about 12 min).

In a fourth aspect of the invention there is provided a bolus dosing system comprising a first IV bolus 15O-water and a second IV bolus 15O-water, for use in a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/VT (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),

wherein the start of the two PET acquisitions of steps a) and c) are interspaced by at least 6 half-lives of 15O (about 12 min).

### DEFINITIONS

In the context of the present application the following definitions will be used:
CVR=Cerebrovascular reserve capacity
CBF=Cerebral blood flow
TIA=Transient ischemic attack
PET=Positron emission tomography
TACs=Time-activity curves
VT=water partition coefficient

15O-water or 15O-H2O means oxygen-15 labelled water which is a radioactive variation of regular water in which the oxygen atom has been replaced by oxygen-15 (15O), a positron-emitting isotope. Oxygen-15 decays with a half-life of about 2.04 minutes to nitrogen-15, emitting a positron. The positron quickly annihilates with an electron, producing two gamma rays of about 511 keV which are detectable using a PET scanner.

The acetazolamide (ACZ) challenge test is a clinical tool used to evaluate cerebrovascular reserve in patients with chronic cerebrovascular disease. It involves administering acetazolamide, a carbonic anhydrase inhibitor, to assess the brain's ability to increase blood flow in response to a vasodilatory stimulus. The ACZ test is is used exclusively for assessment, not for therapeutic purposes, see e.g. Vagal et al. The Acetazolamide Challenge: Techniques and Applications in the Evaluation of Chronic Cerebral Ischemia. AJNR Am J Neuroradiol. 2009 May;30(5):876-884. doi: 10.3174/ajnr.A1538.

### DETAILED DESCRIPTION

State of the art when measuring CBF and CVR requires the use of an arterial input function using arterial cannulation. Many publications (e.g. Fung 2013, Okazawa 2018, Khalighi 2018, Kuttner 2021, Zanotti-Fregonara 2011) have evaluated the use of image-derived input functions, where the arterial input function is estimated directly from the PET images. This is hampered by the limited spatial resolution of PET which is comparable to the dimensions of the carotid arteries and prohibits accurate quantification. None of these methods have yet shown a satisfactory agreement and correlation with arterial sampling. In addition, the proposed methods are highly scanner-and image reconstruction algorithm dependent and cannot be generally applied. The use of arterial spin labelling (ASL) or dynamic susceptibility-weighted (DSC) contrast enhanced magnetic resonance imaging have been suggested for measurement of CVR, but thus far, agreement between ASL and 15O-water has not been high, and DSC requires injection with Gd-based contrast agents which is not tolerated well in all patients.

As described in the summary of invention, the inventors have now surprisingly developed a method for obtaining quantitative CBF and CVR values comprising two 15O-water PET scans, which method obviates the need for arterial cannulation and sampling. The kinetics of 15O-water in brain tissue at baseline can be described by:

$$\frac{dC ( t )}{dt} = F \times {C}_{A} ( t ) - \frac{F}{{V}_{T}} C ( t ) \qquad ( eq . 1 )$$

Here, C(t) is the radioactivity concentration in tissue (regions, voxels) over time, the so-called time-activity curve (TAC); F is CBF, CA(t) is the arterial input curve, and VT is the partition coefficient of water.

The solution of this equation is:

$$C ( t ) = F \times {C}_{A} ( t ) ⊗ {e}^{- \frac{F}{{V}_{T}} t} \qquad ( eq . 2 )$$

By performing two measurements, at baseline and during vasodilation and fitting eq. 2, adding a fitted blood volume parameter, to each dataset separately, F1 (baseline) and F2 (during vasodilation) can be estimated.

Cerebrovascular reserve is then calculated as the ratio of vasodilated and baseline flow:

$$CVR = \frac{{F}_{2}}{{F}_{1}} \qquad ( eq . 3 )$$

Rearranging the differential equations describing the tracer kinetic analysis of 150-water at baseline and during vasodilation yields, when injection of the radioactivity is done in a controlled and reproducible manner, a solution where the arterial input function is no longer required. Instead, the vasodilation data is described as a function of the baseline data and CVR, or vice-versa. Together with a controlled automated power injector, injecting a fixed amount of radioactivity at a fixed injection rate, the method allows for non-invasive measurement of CVR and CBF using 15O-water and PET:

$$Using {F}_{2} = {F}_{1} \times CVR \qquad ( eq . 4 )$$

eq. 2 can be rewritten as:

$$\frac{{dC}_{2} ( t )}{dt} = {F}_{1} \times CVR \times {C}_{A} ( t ) - \frac{{F}_{1} \times CVR}{{V}_{T}} {C}_{2} ( t ) \qquad ( eq . 5 )$$

Multiplying eq. 1 with CVR gives:

$$CVR \times \frac{{dC}_{1} ( t )}{dt} = CVR \times {F}_{1} \times {C}_{A} ( t ) - CVR \times \frac{{F}_{1}}{{V}_{T}} \times {C}_{1} ( t ) \qquad ( eq . 6 )$$

Assuming an identical arterial input function CA(t) during both scans, subtraction of eq. 6 from eq. 5 yields:

$$\frac{{dC}_{2} ( t )}{dt} - CVR \times \frac{{dC}_{1} ( t )}{dt} = \frac{{F}_{1} \times CVR}{{V}_{T}} \times {C}_{1} ( t ) - \frac{{F}_{1} \times CVR}{{V}_{T}} \times {C}_{2} ( t ) \qquad ( eq . 7 )$$

which results in a differential equation for C2(t) that no longer includes CA(t):

$$\frac{{dC}_{2} ( t )}{dt} = CVR \times \frac{{dC}_{1} ( t )}{dt} + \frac{{F}_{1} \times CVR}{{V}_{T}} \times {C}_{1} ( t ) - \frac{{F}_{1} \times CVR}{{V}_{T}} \times {C}_{2} ( t ) \qquad ( eq . 8 )$$

This equation has the following analytical solution:

$${C}_{2} ( t ) = CVR \times {C}_{1} ( t ) + \frac{{F}_{1} \times CVR}{{V}_{T}} \times ( 1 - CVR ) \times {C}_{1} ( t ) ⊗ {e}^{\frac{{F}_{1} \times CVR}{{V}_{T}} t} \qquad ( eq . 9 )$$

Hence, the time-activity curve during vasodilation C2(t) can be described as a function of the baseline time-activity curve C1(t) and the three parameters F1, CVR and VT.

Substituting C2 by C1, F1 and C1 by F2 and C2, respectively, and CVR by 1/CVR, C1(t) can in a similar way be described as a function of C2(t), F and VT. Since F and VT always appear as F/VT in the operational equation, fitting both of them is redundant and instead the system is reduced to a two-parameter solution.

Fitting eq. 9 to the measured tissue TAC during vasodilation using non-linear regression gives CVR and F/VT.

By assuming a fixed value for VT, both F1 and F2 can be separately estimated.

Using this method at the single voxel level is time-consuming due to the required computing power, but eq. 8 can be integrated leading to a linear problem:

$${C}_{2} ( t ) = CVR \times {C}_{1} ( t ) + \frac{{F}_{1} \times CVR}{{V}_{T}} {\int }_{0}^{t} ( {C}_{1} ( τ ) - {C}_{2} ( τ ) ) d τ \qquad ( eq . 10 )$$

$$\frac{{C}_{2} ( t )}{{C}_{1} ( t )} = CVR + \frac{\frac{{F}_{1} \times CVR}{{V}_{T}} {\int }_{0}^{{t}_{( {C}_{1} ( τ ) - {C}_{2} ( τ ) ) d τ}}}{{C}_{1} ( t )} \qquad ( eq . 11 )$$

Evaluating the left-hand side and the integral part of the right-hand side for each image frame and plotting them against each other results in a curve through which a straight line with intercept CVR and slope F1CVR/VT can be fitted. This procedure can be implemented efficiently using generalized least squares and is suitable for performing the analysis on all pixels of a dynamic image set, resulting in parametric images showing CVR at the voxel level.

In addition, a basis function implementation of equation 9 can be implemented, which also allows for linearization of the solution and fast computation of parametric images.

Eq. 9 can be rewritten as:

$${C}_{2} ( t ) = {θ}_{1} \times {C}_{1} ( t ) + {θ}_{2} \times {C}_{1} ( t ) ⊗ {e}^{- {θ}_{3} t} \qquad ( eq . 12 )$$

$$where {θ}_{1} = CVR , {θ}_{2} = \frac{{F}_{1} \times CVR}{{V}_{T}} \times ( 1 - CVR ) and {θ}_{3} = \frac{{F}_{1} \times CVR}{{V}_{T}} .$$

A set of basis functions BFi can then be created using a pre-defined discrete set of θ₃ values

$${BF}_{i} ( t ) = {C}_{1} ( t ) ⊗ {e}^{- {θ}_{3 , i} t} \qquad ( eq . 13 )$$

and eq. 12 can be rewritten as a linear equation for each basis function:

$${C}_{2} ( t ) = {θ}_{1} \times {C}_{1} ( t ) + {θ}_{2} \times {BF}_{i} ( t ) \qquad ( eq . 14 )$$

Equation 14 can then be solved using linear least squares for each basis function BFi, and the basis function for which the residual sum of squares is lowest defines which of θ₁, θ₂ and θ₃ best describes the measured PET data. From these, CVR and F can then be determined. A typical preselected set of θ₃ values would compare 100 values ranging from 0.1 to 2 min−1.

The present disclosure thus relates to the non-invasive estimation of the cerebrovascular blood flow (CBF) and cerebrovascular reserve (CVR) in a human subject.

Accordingly, in a first aspect of the invention there is provided a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/VT (where VT is the water partition coefficient) and CVR,
g) returning estimated (maximum-likelihood) parameter values of the pharmacokinetic model for the brain including cerebral blood flow (CBF) and an estimate of the cerebrovascular reserve (CVR),
   wherein the start of the two PET acquisitions of steps a) and c) are interspaced by at least 6 half-lives of 15O (about 12 min).

The present disclosure also relates to acetazolamide for use in a a non-therapeutic and non-invasive method for the estimation of the cerebrovascular blood flow (CBF) and cerebrovascular reserve (CVR) in a human subject.

In a second aspect of the invention acetazolamide is therefore provided for use in a non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/VT (where VT is the water partition coefficient) and CVR,
g) returning estimated (maximum-likelihood) parameter values of the pharmacokinetic model for the brain including cerebral blood flow (CBF) and an estimate of the cerebrovascular reserve (CVR),
   wherein the start of the two PET acquisitions of steps a) and c) are interspaced by at least 6 half-lives of 15O (about 12 min).

The present disclosure also relates to a bolus composition comprising a first IV bolus 15O-water and a second IV bolus 15O-water for use in a non-therapeutic method for non-invasive quantitative measurement of the cerebrovascular blood flow (CBF) and cerebrovascular reserve (CVR) in a human subject.

In a third aspect of the invention there is therefore provided a bolus composition comprising a first IV bolus 15O-water and a second IV bolus 15O-water, for use in a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/V_{T} (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),

wherein the start of the two PET acquisitions of steps a) and c) are interspaced by at least 6 half-lives of 15O (about 12 min).

Finally, the present disclosure also relates to a bolus dosing system comprising a first IV bolus 15O-water and a second IV bolus 15O-water for use in a non-therapeutic method for non-invasive estimation of the cerebrovascular blood flow (CBF) and cerebrovascular reserve (CVR) in a human subject.

In a fourth aspect of the invention there is therefore provided a bolus dosing system comprising a first IV bolus 15O-water and a second IV bolus 15O-water, for use in a method for non-invasive quantitative measurement of cerebrovascular reserve (CVR) and cerebral blood flow (CBF) in a human, which method comprises the following steps:
a) administering a first IV bolus ¹⁵O-water to a human subject and simultaneously starting a 1^{st} PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into dynamic series using all appropriate corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/V_{T} (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),

wherein the start of the two PET acquisitions of steps a) and c) are interspaced by at least 6 half-lives of 15O (about 12 min).

In an embodiment of any of the aspects of the present invention the first and second IV bolus 15O-water are identical. In an embodiment each IV bolus 15O-water contains an activity of 400±40 MBq. In a preferred embodiment each IV bolus 15O-water contains an activity of 400±3 MBq.

In an embodiment of any of the aspects of the present invention the start of the two PET acquisitions of steps a) and c) are interspaced by at least 12 minutes, such as by 12 minutes, by 14 minutes, by 16 minutes or by at least 20 minutes.

In an embodiment of any of the aspects of the present invention the first and

second IV bolus 15O-water each has a volume of 2 ml±0.1 ml. In a preferred embodiment the first and second IV bolus 15O-water each have a volume of 5 ml±0.5 ml.

In a preferred embodiment of any of the aspects of the present invention the first and second IV bolus 15O-water are each administered over 5±1 seconds.

In a preferred embodiment of any of the aspects of the present invention each IV bolus 15O-water is administered as 5 mL±0.5 ml 15O-water at an injection speed of 1 mL/s followed by 35 mL±1 ml saline at an injection speed of 2 mL/s, using a power injector.

In a preferred embodiment the method steps a)-g) according to any aspect of the present invention are not carried out on a human subject that suffers from an acute medical condition.

In another preferred embodiment, the method according to any aspect of the present invention is carried out on a human subject with an aim to obtain a quantitative measurement of cerebrovascular reserve (CVR) and cerebral blood flow (CBF) of said human subject, provided said human subject does not have an acute medical condition, either diagnosed or symptomatic.

In a preferred embodiment of any of the aspects of the present invention the method for non-invasive quantitative measurement of cerebrovascular reserve (CVR) and cerebral blood flow (CBF) in a human is furthermore non-therapeutic.

In another preferred embodiment, the method according to any aspect of the present invention is carried out on a human subject with an aim to obtain a quantitative measurement of cerebrovascular reserve (CVR) and cerebral blood flow (CBF) of said human subject, provided said human subject is not experiencing an acute medical condition at the time said method is scheduled to be carried out.

In yet another preferred embodiment, the method according to any aspect of the present invention is carried out on a human subject with an aim to obtain a quantitative measurement of cerebrovascular reserve (CVR) and cerebral blood flow (CBF) of said human subject, provided said human subject does not experience or have an acute medical condition, either diagnosed or symptomatic, selected from acute mountain sickness, acute angle-closure glaucoma, acute edema, acute seizures such as acute epileptic seizures, and acute increased intracranial pressure, concurrent with, or during the performance of said method.

In another preferred embodiment of any of the aspects of the present invention azetazolamide is administered as a single IV bolus injection of 10 mg/kg body weight.

In another embodiment of any of the aspects of the present invention azetazolamide is replaced by another vasodilatant, preferably another carbonic anhydrase inhibitor.

In an embodiment of any of the aspects of the present invention, the PET scans following administration of the first and second bolus 15O-water are each performed as dynamic PET scans of the brain of a duration of 10 min±2 min.

In a preferred embodiment of any of the aspects of the present invention, the PET scans following administration of the first and second bolus 15O-water are each performed as dynamic PET scans of the brain, each having a duration of 4-10 min.

In another embodiment of any of the aspects of the present invention the dynamic series reconstruction comprises frames of 1×10, 8×5, 4×10, 2×15, 3×20, 2×30, 2×60 seconds duration.

In a preferred embodiment of any of the aspects of the present invention the extracted TACs are fitted to the following pharmacokinetic model (eq. 9):

$${C}_{2} ( t ) = CVR \times {C}_{1} ( t ) + \frac{{F}_{1} \times CVR}{{V}_{T}} \times ( 1 - CVR ) \times {C}_{1} ( t ) ⊗ {e}^{\frac{{F}_{1} \times CVR}{{V}_{T}} t}$$

wherein C2(t) is the time-activity curve (TAC) during vasodilation, C1(t) is the baseline TAC, F1 is the CBF at baseline (i.e. CBF1), VT is the water partition coefficient and CVR is the cardiovascular reserve.

Or, using F1=CBF1,

$${C}_{2} ( t ) = CVR \times {C}_{1} ( t ) + \frac{{CBF}_{1} \times CVR}{{V}_{T}} \times ( 1 - CVR ) \times {C}_{1} ( t ) ⊗ {e}^{\frac{C B {F}_{1} \times CVR}{{V}_{T}} t}$$

In Eq.9, substituting C2 by C1, F1 and C1 by F2 and C2, respectively, and CVR by 1/CVR, leads to C1(t) in a similar way can be described as a function of C2 t), F2 and VT.

In another embodiment of any of the aspects of the present invention the extracted TACs are fitted to the following pharmacokinetic model:

$${C}_{1} ( t ) = 1 / CVR \times {C}_{2} ( t ) + \frac{{CBF}_{2} \times CVR}{{V}_{T}} \times ( 1 - CVR ) \times {C}_{2} ( t ) ⊗ {e}^{\frac{{CBF}_{2} \times CVR}{{V}_{T}} t}$$

Alternatively to steps e) and f) of any of the aspects of the present invention, fit individual corresponding voxel TACs to the same tracer kinetic model or a linearization of this model to obtain maps of CBF/VT and CVR.

Steps a)-g) of any of the aspects of the present invention can conveniently be automated by employing a computing device comprising a computer readable storage medium containing instructions that, when executed by a processor, are configured to cause said computing device to execute or perform a method comprising said steps a)-g).

The general, or overall methods referred to above are based on a plurality of emission tomography images, such as positron emission tomography (PET) images wherein each image represents concentrations of a tracer that has been injected at a specific time. The PET system detects radiation emitted indirectly by the tracer, i.e. after a tracer has been injected in the blood flow, a time activity curve (TAC) can be obtained by observing the radiation in a pixel/voxel over time.

PET is the preferred imaging method according to the preferred embodiments of the invention.

The present disclosure further relates to a system for estimating the cerebrovascular reserve of a human based on two sets of a plurality of emission tomography images, such as positron emission tomography (PET) images of the brain, wherein the acquisition of the two sets of e.g. PET images is interspaced by the administration of a vasodilating agent to said human, such that the first set of e.g. PET images is acquired under normal (“baseline”) conditions and the second set under conditions where the healthy intracranial arteries are dilated, said system being arranged to perform the method according to the method described in the present disclosure.

By comparison of the two sets of e.g. PET images, regions of the brain which are supplied by stenotic or malformed blood vessels (which do not dilate significantly in response to the administered vasodilating agent) can be mapped/quantified. The cardiovascular reserve is next calculated as the ratio of vasodilated and baseline flow.

This means that preferably the system comprises an emission imaging device arranged to provide the emission tomography images; and a processor or other means arranged to perform the method for mapping and/or identifying regions of the brain which are supplied by stenotic or malformed blood vessels.

Furthermore, a non-transitive, computer-readable storage device for storing instructions that, when executed by a processor, performs the various methods described hereinabove according to the present disclosure. The methods may be implemented as software that is readable and executable by for example a computer processor.

### EXPERIMENTAL

After filing the priority document EP24178667.2 applicant have found that 2subjects had been mis-labeled as multiple-sclerosis patients, so that the data set of EP24178667.2comprised 4 healthy control subjects and 8 multiple-sclerosis patients. Applicant have since added 5 healthy control subjects (bringing the total to 9 healthy controls+8 multiple-sclerosis patients) which are included in the below updated experimental section.

### Clinical Data—Comparison Invasive vs. Non-Invasive

Seventeen subjects (9 healthy controls, 8 multiple-sclerosis patients) underwent 15O-water PET-CT scans at baseline and during acetazolamide challenge (10 mg/kg). Both scans consisted of a 10 min dynamic scan after administration of 400±40 MBq 15O-water using a power injector (5 mL @ 1 mL/s followed by 35 mL saline @ 2 mL/s). Arterial blood was sampled continuously and measured using an on-line detector (Veenstra PBS 101, Comercer, Joure, The Netherlands, 14 subjects, or Twilite Two, Swisstrace, Zurich, Switzerland). Images were corrected for frame-by-frame motion and volumes of interest were defined on a co-registered T1-MRI using a probabilistic template (PVElab) and transferred to all PET images to create time-activity curves. Each scan was then analysed separately using non-linear regression of the single-tissue compartment model with delay-and dispersion-corrected arterial input function and fitted blood volume parameter, as well as using the proposed non-invasive model. As the exact amounts of injected activity were not known, the post-acetazolamide time-activity curves were scaled by the ratio of the areas under the curve of the first-pass phases of the baseline and post-acetazolamide arterial input functions to correct for possible differences in injected activity.

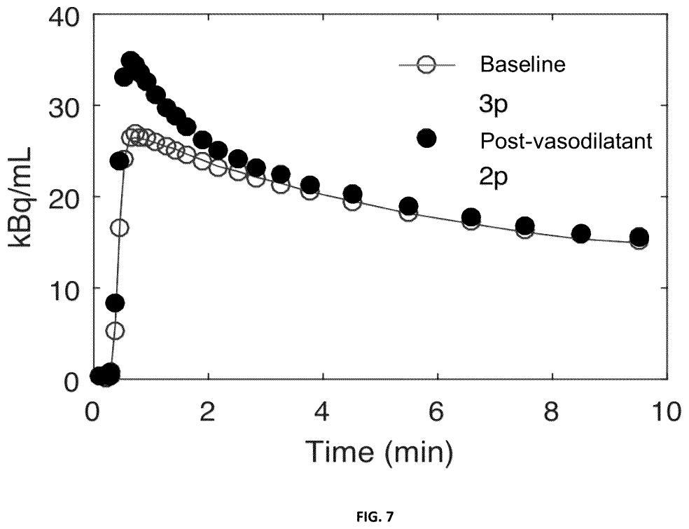

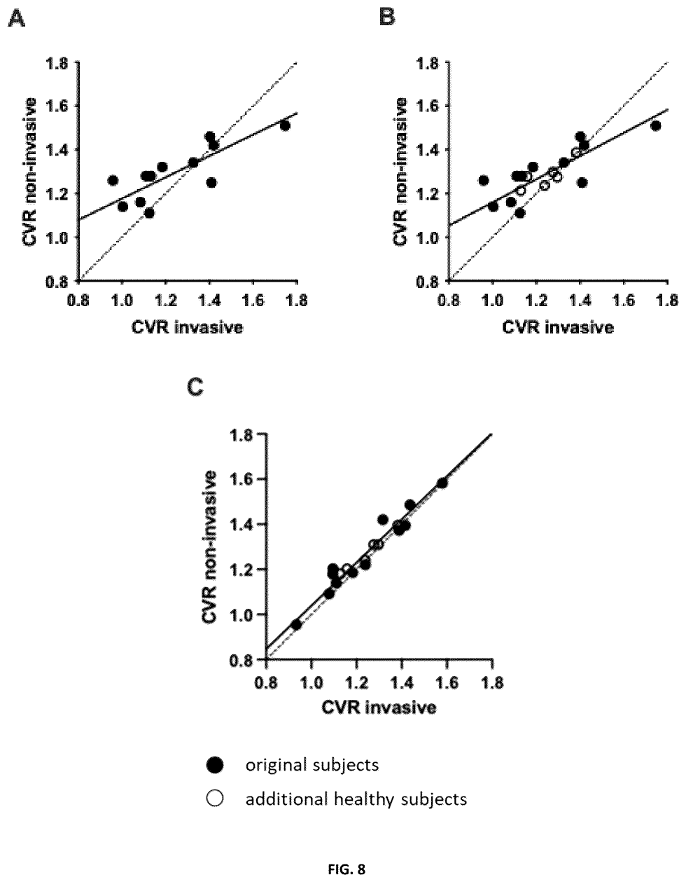

FIG. 7 shows a typical fit of the proposed model to a total brain grey matter time-activity curve and FIG. 8 shows the relation between CVR based on separate arterial plasma-input model fits and CVR estimated using the proposed method, both without (A) and after (B) scaling of the post-acetazolimide time-activity curves as described above.

### Simulations

One hundred baseline time-activity curves (TACs) were simulated using equation (2) above, with CBF values chosen randomly in the interval between 0.3 and 0.7 mL/g/min and VT fixed to 0.85 mL/g. One hundred corresponding TACs after vasodilation were added using CVR chosen randomly between 1.0 and 1.6. Each pair of TACs was then analysed using equation 9, both with 3 and 2 parameters, and using equation 11 for the interval between 1 and 4min after injection.

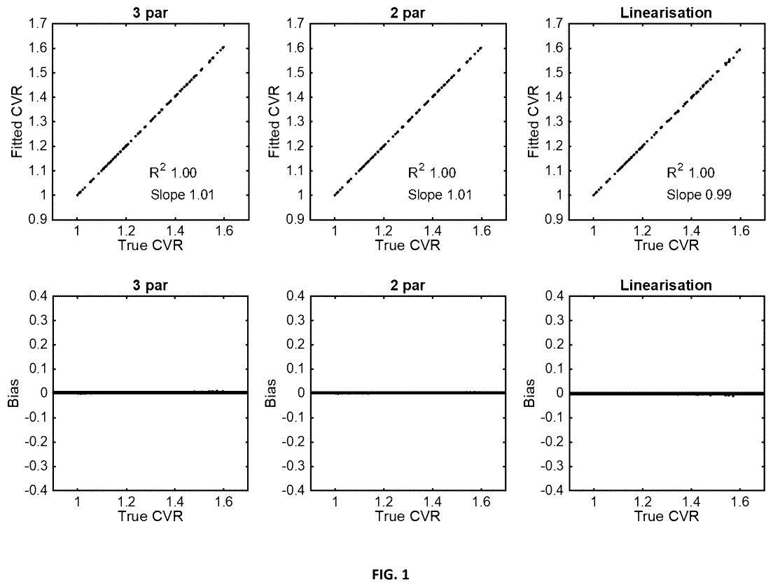

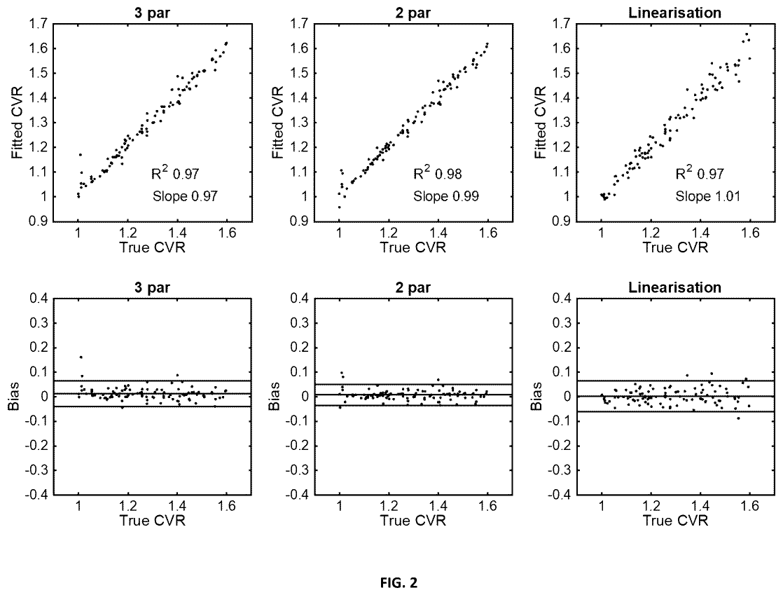

As shown in FIG. 1, both 2- and 3-parameter models and the linearised version of the model produce nearly exactly the correct CVR when all assumptions are fulfilled, and data is noise-free. A random noise level typical of regional 15O-water PET data in the brain was added independently to each simulated TAC, and the simulation was repeated. Each pair of TACs was then again analysed using equation 9, both using 3 and 2 parameters, and using equation 11. As shown in FIG. 2, both 2- and 3-parameter models result in an excellent correlation and agreement between true and estimated CVR.

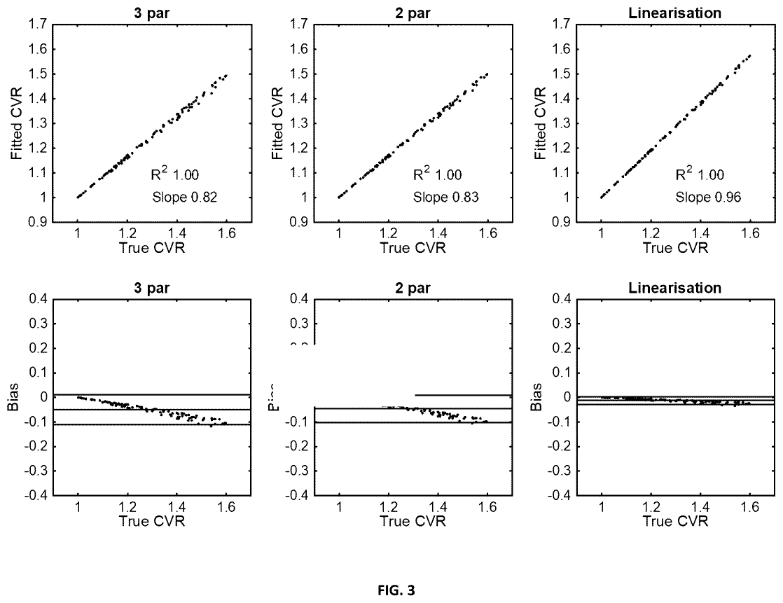

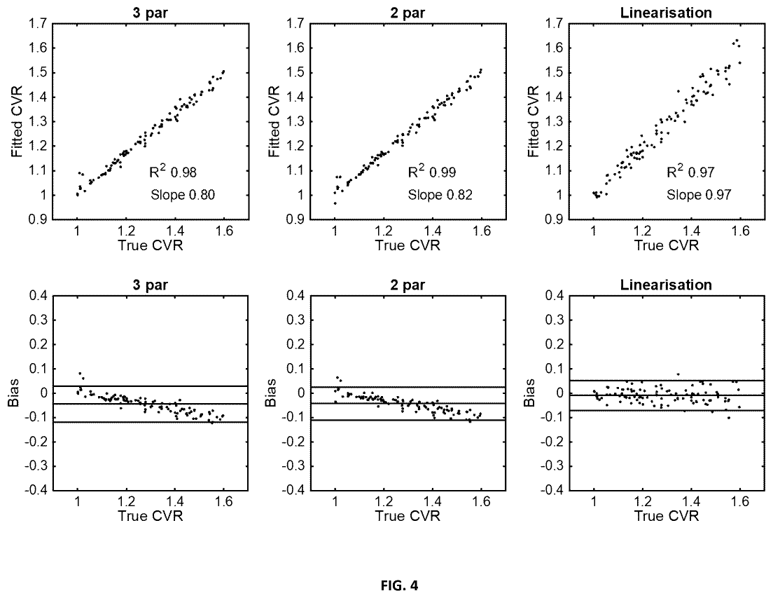

Effect of partial blood volume: Then, a typical blood volume of 5% was added and the simulations were repeated for noise-free (FIG. 3) and noisy (FIG. 4) data. As apparent in FIG. 3, blood volume creates a negative bias proportional to CVR itself, but correlation is not affected. In addition, the linearised model appears to be affected less by blood volume than the full model.

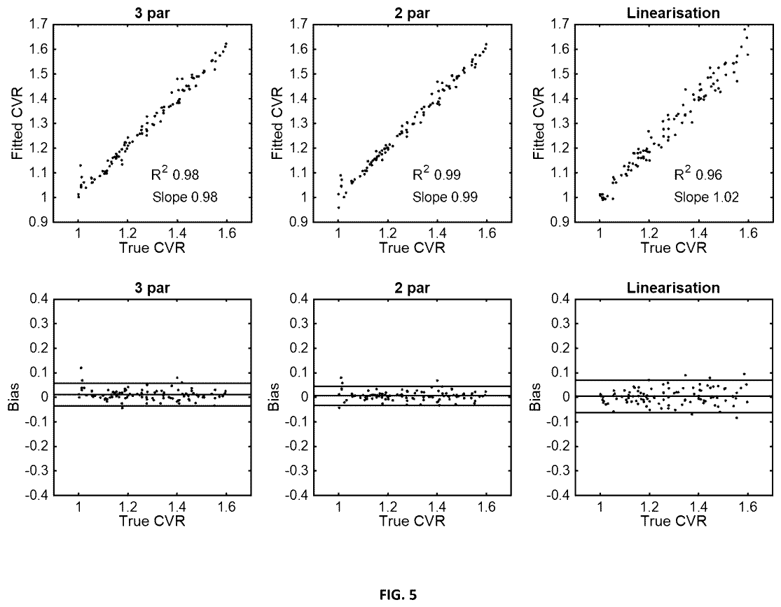

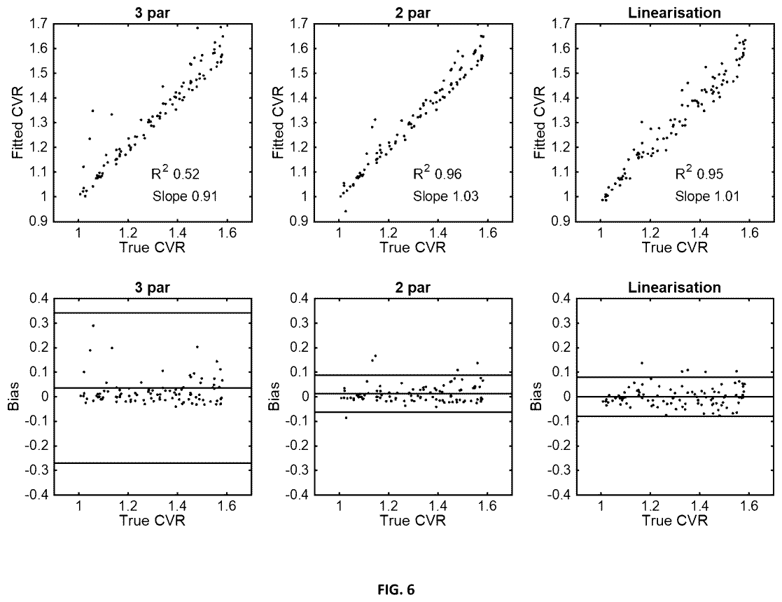

Correction for partial blood volume: FIGS. 5 and 6 represent two different ways of correcting for a blood volume component. First, the assumption was made that a noisy representation of the true input curve can be extracted from the PET data itself (e.g., using the carotid artery TAC). This noisy representation of the input curve was subtracted from the simulated tissue TACs prior to analysis, assuming a 5% blood volume, resulting in the correlations and biases shown in FIG. 5. Second, another possible correction would be to subtract a population-average arterial input curve from the tissue TACs prior to analysis. To this end, the simulation was repeated with a different input curve for each simulated subject, with a ±20% variation in peak height, ±20% variation in peak clearance, and ±10% variation in tail clearance. This resulted in the correlations and biases shown in FIG. 6.

### REFERENCES

1. Mamontov et al. Animal model of assessing functional reserve by imaging photoplethysmography. Nature Scientifc Reports|(2020) 10:19008| https://doi.org/10.1038/s41598-020-75824-w
2. Farzam K et al. Acetazolamide. [Updated 2023 Jul. 2]. In: StatPearls [Internet]. Treasure Island (FL): StatPearls Publishing; 2025 January-. Available from: https://www.ncbi.nlm.nih.gov/books/NBK532282/
3.Vagal et al. The Acetazolamide Challenge: Techniques and Applications in the Evaluation of Chronic Cerebral Ischemia. AJNR Am J Neuroradiol. 2009 May: 30(5):876-884. doi: 10.3174/ajnr.A1538
4. Gupta et al. Cerebrovascular Reserve and Stroke Risk in Patients with Carotid Stenosis or Occlusion. Stroke November 2012 p.2884 https://doi.org/10.1161/STROKEAHA.112.663716
5. Everett et al. Safety of Radial Arterial Catheterization in PET Research Subjects. J Nucl Med. 2009 October; 50(10): 1742. https://doi.org/10.2967/jnumed.109.063206
6. Acker et al. ¹⁵O-WATER PET IN NEGATIVE ^{99M}Tc-HMPAO SPECT. The Journal of Nuclear Medicine • Vol. 59 • No. 2 • February 2018
7. Chim et al. Complications related to radial artery occlusion, radial artery harvest, and arterial lines. Hand Clin. 2015;31(1):93-100.
8. Mandel et al. Radial artery cannulation and complications in 1,000 patients: precautions. J Hand Surg. 1977;2(6):482-5.
9. Wallach et al. Cannulation injury of the radial artery: diagnosis and treatment algorithm. Am J Crit Care Off Publ Am Assoc Crit-Care Nurses. 2004; 13 (4):315-9.
10. Everett et al. Safety of radial arterial catheterization in PET research subjects. J Nucl Med Off Publ Soc Nucl Med. 2009;50(10):1742.
11. Fung et al. Cerebral blood flow with [¹O]water PET studies using an image-derived input function and MR-defined carotid centerlines. Phys Med Biol. 2013;58 (6):1903-23.
12. Okazawa et al. Noninvasive method for measurement of cerebral blood flow using O-15water PET/MRI with ASL correlation. Eur J Radiol. 2018;105:102-9
13. Khalighi et al. Image-derived input function estimation on a TOF-enabled PET/MR for cerebral blood flow mapping. J Cereb Blood Flow Metab. 2018;38(1):126-35.
14. Kuttner et al. Cerebral blood flow measurements with ¹⁵O-water PET using a non-invasive machine-learning-derived arterial input function. J Cereb Blood Flow Metab. 2021;6:66.
15. Zanotti-Fregonara et al. Image-derived input function for brain PET studies: many challenges and few opportunities. J Cereb Blood Flow Metab Off J Int Soc Cereb Blood Flow Metab. 2011;31(10):1986-98 .

1. A method for non-invasive quantitative measurement of cerebrovascular reserve (CVR) and cerebral blood flow (CBF) in a human subject, which method comprises the following steps:
a) administering a first IV bolus ¹⁵O-water to said human subject and simultaneously starting a 1st PET acquisition of the brain, followed by
b) administering a single bolus of acetazolamide to said human subject, 5-15 minutes before the second injection of ¹⁵O-water, followed by
c) administering a second IV bolus ¹⁵O-water to said human subject and simultaneously starting a 2^{nd} PET acquisition of the brain, followed by
d) reconstructing both PET acquisitions into a dynamic series using corrections for quantitative PET, followed by
e) measuring the activity concentrations in identical regions of interest in both scans and construct time-activity curves (TACs) showing the activity concentration over time, followed by
f) fitting the combinations of TACs to a tracer kinetic model that describes one of the TACs as a function of the other, CBF/V_{T} (where V_{T} is the water partition coefficient) and CVR,
g) returning estimated (maximum-likelihood) parameter values of the pharmacokinetic model for the brain including cerebral blood flow (CBF) and an estimate of the cerebrovascular reserve (CVR),
wherein the start of the two PET acquisitions of steps a) and c) are interspaced by at least 6 half-lives of ¹⁵O (about 12 min).

2. The method according to claim 1, wherein said human subject does not have or experience an acute medical condition concurrent with, or during the performance of the method of claim 1.

3. The method according to claim 2, wherein said acute medical condition is selected from acute mountain sickness, acute angle-closure glaucoma, acute edema, acute seizures such as acute epileptic seizures, and acute increased intracranial pressure.

4. The method according to claim 1, wherein acetazolamide is administered as an IV dose of 10 mg/kg body weight.

5. The method according to claim 1, wherein the first and the second IV bolus ¹⁵O-water contain the same activity.

6. The method according to claim 1, wherein each IV bolus ¹⁵O-water contains an activity of 400±3 MBq.

7. The method according to claim 1, wherein each IV bolus ¹⁵O-water has a volume of 5 ml ±0.5 ml.

8. The method according to claim 1, wherein each bolus ¹⁵O-water is administered over 5±1 seconds.

9. The method according to claim 1, wherein each IV bolus ¹⁵O-water is administered as 5mL±0.5 ml ¹⁵O-water at an injection speed of 1 mL/s followed by 35 mL±1 ml saline at an injection speed of 2 mL/s, using a power injector.

10. The method according to claim 1, wherein azetazolamide is replaced by another carbonic anhydrase inhibitor.

11. The method according to claim 1, wherein the PET scans following administration of the first and second bolus ¹⁵O-water are each performed as dynamic PET scans of the brain of a duration of 10 min±2 min.

12. The method according to claim 1, wherein the dynamic series reconstruction comprises frames of 1×10, 8×5, 4×10, 2×15, 3×20, 2×30, 2×60 seconds duration.

13. The method according to claim 1, wherein the corrections for quantitative PET are selected from decay, scatter, and dead-time.

14. The method according to claim 1, wherein the time-activity curves (TACs) are fitted to a pharmacokinetic model:
$${C}_{2} ( t ) = CVR \times {C}_{1} ( t ) + \frac{{F}_{1} \times CVR}{{V}_{T}} \times ( 1 - CVR ) \times {C}_{1} ( t ) ⊗ {e}^{\frac{{F}_{1} \times CVR}{{V}_{T}} t}$$
wherein C₂(t) is the TAC during vasodilation, C₁(t) is the baseline TAC, F₁ is the cerebral flow at baseline (i.e. CBF₁), V_{T} is the water partition coefficient and CVR is the cardiovascular reserve.

15. The method according to claim 1, wherein the method is characterized in being non-therapeutic.
